(19) United States
(12) Patent Application Publication
(10) Pub. No.: US 2024/0368048 A1
(43) Pub. Date: Nov. 7, 2024

(54) COMPOSITIONS AND METHODS FOR SILICA GEL ENCAPULATION OF PLANT GROWTH PROMOTING BACTERIA

(71) Applicant: THE REGENTS OF THE UNIVERSITY OF CALIFORNIA, Oakland, CA (US)

(72) Inventors: Anne M. HIRSCH, Los Angeles, CA (US); Chong LIU, Los Angeles, CA (US); Luke ELISSIRY, Los Angeles, CA (US)

(73) Assignee: THE REGENTS OF THE UNIVERSITY OF CALIFORNIA, Oakland, CA (US)

(21) Appl. No.: 18/683,186

(22) PCT Filed: Aug. 12, 2022
(86) PCT No.: PCT/US2022/074924

§ 371 Date: Feb. 12, 2024

Related U.S. Application Data
(60) Provisional application No. 63/232,505, filed on Aug. 12, 2021.

(51) Int. Cl. C05F 11/08; C05D 9/00; C05G 5/30

(52) U.S. Cl. CPC C05F 11/08; C05D 9/00; C05G 5/30

(57) ABSTRACT

Methods and compositions for silica gel encapsulation of plant growth-promoting bacteria are disclosed. Aspects of the disclosure include silica gel microbeads comprising one or more plant growth-promoting bacteria. Also disclosed are methods for generating bacteria-containing silica gel microbeads. Further described are methods for promoting plant growth comprising providing a bacteria-containing silica based solution (e.g., droplets) to a plant seed. Plant seeds coated with bacteria-containing silica gel microbeads are disclosed herein.

### CROSS REFERENCE TO RELATED APPLICATIONS

This application claims benefit of priority to U.S. Provisional Application No. 63/232,505, filed Aug. 12, 2021, which is hereby incorporated by reference in its entirety.

### STATEMENT OF GOVERNMENT SUPPORT

This invention was made with government support under Grant Number GM055052, awarded by the National Institutes of Health. The government has certain rights in the invention.

### BACKGROUND

### I. Field of the Invention

Aspects of this invention relate to at least the fields of inorganic chemistry, microbiology, and plant biology.

### II. Background

Synthetic fertilizer is responsible for the greatly increased crop yields that have enabled worldwide industrialization. However, the production and use of such fertilizers are environmentally unfriendly and unsustainable; synthetic fertilizers are produced via non-renewable resources and fertilizer runoff causes groundwater contamination and eutrophication. A promising alternative to synthetic fertilizer is bacterial inoculation. In this process, a symbiotic relationship is formed between a crop and bacteria species that can fix nitrogen, solubilize phosphorus, and stimulate plant hormone production. Bacterial inoculation is an alternative to synthetic fertilizers due to some species of microbes' inherent abilities to fix nitrogen, solubilize complexed phosphorus, induce resistance to abiotic and biotic stress, and stimulate plant hormone production. However, plant growth-promoting bacteria (PGPB) must compete with less-efficacious, native microbes that are often better-adapted to the heterogeneous soil microbiome in order to colonize the root system of a plant.

Providing a more suitable and protected microenvironment through immobilization of the desired PGPB is essential to maintaining their viability and effectiveness before they can colonize the root system. PGPB carriers that allow for inoculant delivery and adequate protection are needed to ensure that PGPB remain viable in soil and are available to crops. There exists a need for effective, inexpensive, non-hazardous, and scalable PGPB carriers and methods of use for promoting plant growth.

### SUMMARY

Aspects of the present disclosure provide novel silica gel microbead compositions, as well as methods for making and using such compositions, for example, in encapsulating plant-growth promoting bacteria and promoting plant growth.

Aspects of the disclosure include silica-based solutions, silica gel microbeads, plant seeds, bacterial compositions, methods for generating silica gel microbeads, methods for encapsulating plant growth-promoting bacteria, and methods for promoting plant growth. Compositions of the disclosure may comprise one or more of: silica gel microbeads, plant growth-promoting bacteria, and plant seeds. Silica based solutions of the disclosure can comprise at least 1, 2, 3, or more of: sodium silicate, citric acid, colloidal silica, glycerol, and plant growth-promoting bacteria. One or more of the preceding components may be excluded from solutions of the disclosure. Methods of the disclosure can include at least 1, 2, 3, 4, or more of the following steps: providing a bacteria-containing silica-based solution, providing a silica solution, providing a bacteria solution, mixing a silica solution and a bacteria solution, aerosolizing a solution to form droplets, allowing droplets to gel to form microbeads, coating a plant seed, rinsing a plant seed, and planting a plant seed. One or more of the preceding steps may be specifically excluded from aspects of the disclosure.

Disclosed herein, in some aspects, is a method of increasing one or more plant growth characteristics in a plant, the method comprising (a) providing to a plant seed a solution comprising sodium silicate, citric acid, colloidal silica, and one or more plant growth-promoting bacteria; and (b) rinsing the plant seed. In some aspects, (b) comprises rinsing the plant seed with water. In some aspects, (b) comprises rinsing the plant seed with phosphate buffered saline. In some aspects, the method further comprises planting the plant seed.

In some aspects, (a) comprises aerosolizing the solution to form droplets. In some aspects, the droplets are less than 400 μm in diameter. In some aspects, the droplets are between about 50 μm and about 200 μm in diameter. In some aspects, the droplets comprise between 10% and 20% silica by weight. The droplets may comprise at least, at most, or about 10, 11, 12, 13, 14, 15, 16, 17, 18, 19, or 20% silica by weight, or any range or value derivable therein. In some aspects, the droplets comprise about 13% silica by weight. In some aspects, the method further comprises, prior to (b), allowing the droplets to gel to form silica microbeads. In some aspects, (b) is performed for at least 15 minutes. In some aspects, (b) is performed at least 5 minutes after (a). In some aspects, the method further comprises rinsing the plant seed a second time.

Further disclosed herein, in some aspects, is a method of making a population of plant-growth promoting silica microbeads, the method comprising (a) aerosolizing a solution comprising sodium silicate, citric acid, colloidal silica, and one or more plant-growth promoting bacteria to generate droplets; (b) allowing the droplets to gel to form silica microbeads; and (c) rinsing the silica microbeads. In some aspects, (c) comprises rinsing the silica microbeads with water. In some aspects, (c) comprises rinsing the silica microbeads with phosphate buffer saline. In some aspects, the droplets are less than 400 μm in diameter. In some aspects, the droplets are between about 50 μm and about 200 μm in diameter. In some aspects, the droplets comprise between 10% and 20% silica by weight. The droplets may comprise at least, at most, or about 10, 11, 12, 13, 14, 15, 16, 17, 18, 19, or 20% silica by weight, or any range or value derivable therein. In some aspects, the droplets comprise about 13% silica by weight. In some aspects, the droplets comprise 13% silica by weight. In some aspects, the method further comprises, prior to (b), allowing the droplets to gel to form silica microbeads. In some aspects, (c) is performed for at least 15 minutes. In some aspects, (c) is performed at least 5 minutes after (b). In some aspects, the method further comprises rinsing the plant seed a second time. In some aspects, the method further comprises planting the plant seed.

In some aspects, the solution comprises between 1.0 and 2.0 mol/L sodium silicate. The solution may comprise at least, at most, or about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mol/L sodium silicate, or any range or value derivable therein. In some aspects, the solution comprises about 1.5 mol/L sodium silicate. In some aspects, the solution comprises 1.5 mol/L sodium silicate. In some aspects, the solution comprises between 0.5 and 1.5 mol/L citric acid. The solution may comprise at least, at most, or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 mol/L citric acid, or any range or value derivable therein. In some aspects, the solution comprises about 1.0 mol/L citric acid. In some aspects, the solution comprises 1.0 mol/L citric acid. In some aspects, the solution comprises a ratio of the colloidal silica to the sodium silicate of between 2.5:4 and 3.5:4. In some aspects, the solution comprises a ratio of the colloidal silica to the sodium silicate of 3:4. In some aspects, the solution is at a pH of between 6.5 and 7.5. The solution may be at a pH of at least, at most, or about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, or any range or value derivable therein. In some aspects, the solution is at a pH of about 7.0. In some aspects, the solution is at a pH of 7.0. In some aspects, the solution comprises at least about 10⁷ CFU/mL of the plant growth-promoting bacteria. The solution may comprise at least, at most, or about 10⁵, 10⁶, 10⁷, 10⁸, or 10⁹ CFU/mL of the plant growth-promoting bacteria, or any range or value derivable therein. In some aspects, the solution comprises at least 10⁷ CFU/mL of the plant growth-promoting bacteria. In some aspects, the solution does not comprise glycerol. In some aspects, the solution comprises glycerol.

In some aspects, the method further comprises, prior to (a), generating the solution by mixing (i) a silica solution comprising the sodium silicate, citric acid, and colloidal silica; and (ii) a bacteria solution comprising the plant growth-promoting bacteria. In some aspects, the solution does not gel less than 1, 2, 3, 4, 5, or more minutes following mixing the silica solution and the bacteria solution. In some aspects, the solution does not gel less than 2 minutes following mixing the silica solution and the bacteria solution. In some aspects, the solution does not gel less than 3 minutes following mixing the silica solution and the bacteria solution. In some aspects, the solution does not gel less than 4 minutes following mixing the silica solution and the bacteria solution.

Further disclosed herein, in some aspects, is a plant seed coated with a population of silica microbeads comprising at least 1000 colony forming units (CFU) of plant-growth promoting bacteria. In some aspects, the plant seed comprises at least 3000 CFU of the plant-growth promoting bacteria. In some aspects, the plant seed comprises at least 5000 CFU of the plant-growth promoting bacteria. In some aspects, the plant seed comprises at least 10000 CFU of the plant-growth promoting bacteria. In some aspects, the plant seed comprises at least 15000 CFU of the plant-growth promoting bacteria. The plant seed may comprise at least, at most, or about 1000, 1500, 2000, 3000, 4000, 5000, 6000, 7000, 8000, 9000, 10000, 15000, 20000, 30000, 40000, or 50000 CFU of plant-growth promoting bacteria. Also disclosed is a population of plant seeds comprising a plant seed of the disclosure.

In some aspects, the one or more plant growth-promoting bacteria comprise Paenibacillus pabuli 151 (NRRL Accession No. B-67417), Dietzia cinnamea 55 (NRRL Accession No. B-67422), Lysinobacillus sphaericus 47 (NRRL Accession No. B-67423), Paenibacillus MBEV37 B17 (Accession No. B-67419), Exiguobacterium alkaliphilum 20 (NRRL Accession No. B-67425), or Bacillus safensis 34 (NRRL Accession No. B-67620), Methylobacterium dankookense Bots301 (NRRL Accession No. B-67981), Methylobacterium radiotolerans Bots303 (NRRL Accession No. B-67982), Fictibacillus phosphorivorans Bots309 (NRRL Accession No. B-67983), Bradyrhizobium japonicum USDA 110, Sinorhizobium meliloti Rm1021, or Micromonospora sp. UTRUM1 (NRRL Accession No. B-67418). In some aspects, the one or more plant growth-promoting bacteria comprise Sinorhizobium meliloti Rm1021. In some aspects, the one or more plant growth-promoting bacteria comprise Bradyrhizobium japonicum USDA 110. In some aspects, the plant seed is a dicotyledon plant seed, a crop plant seed, or a legume plant seed. In some aspects, the plant seed is an alfalfa plant seed.

Further disclosed herein, in some aspects, is a silica microbead encapsulating one or more plant growth-promoting bacteria. In some aspects, the microbead has a diameter of less than 400 μm. In some aspects, the microbead has a diameter of less than 200 μm. In some aspects, the microbead has a diameter of less than 100 μm. In some aspects, the microbead has a diameter of about 50 μm. In some aspects, the microbead is between 10% and 20% silica by weight. In some aspects, the microbead is about 13% silica by weight. In some aspects, the one or more plant growth-promoting bacteria comprise Paenibacillus pabuli 151 (NRRL Accession No. B-67417), Dietzia cinnamea 55 (NRRL Accession No. B-67422), Lysinobacillus sphaericus 47 (NRRL Accession No. B-67423), Paenibacillus MBEV37 B17 (Accession No. B-67419), Exiguobacterium alkaliphilum 20 (NRRL Accession No. B-67425), or Bacillus safensis 34 (NRRL Accession No. B-67620), Bradyrhizobium japonicum USDA 110, or Sinorhizobium meliloti Rm1021. In some aspects, the one or more plant growth-promoting bacteria comprise Sinorhizobium meliloti Rm1021. In some aspects, the one or more plant growth-promoting bacteria comprise Bradyrhizobium japonicum USDA 110. Also disclosed is a population of microbeads comprising a silica microbead of the disclosure. In some aspects, the population of microbeads comprises at least about 10⁷ CFU of the plant growth promoting bacteria. In some aspects, the population of microbeads comprises an additional silica microbead encapsulating an additional plant growth-promoting bacteria. Also disclosed is a plant seed coated with a population of microbeads of the disclosure.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Any aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

It is specifically contemplated that aspects described herein may be excluded. It is further contemplated that, when a range is described, certain ranges may be excluded.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIG. 1 shows a schematic of microbead encapsulation of plant growth promoting bacteria (PGPB). The schematic demonstrates the replacement of environmentally harmful nitrogen fertilizers with symbiotic bacteria encapsulated in a protective silica matrix and applied directly to crop seeds. Silica gel microbeads break down in the soil over time, releasing the immobilized PGPB into the plant root system, where it can fix nitrogen gas (N₂) into utilizable nitrogen, increasing crop yields.

FIG. 2 shows a diagram detailing bacteria-containing silica gel microbead production. Silica sol is combined with the culture solution to produce an inorganic/biological hybrid sol. The sol is homogenized before being added to an atomizer and sprayed directly onto seeds. This process produces microbead droplets (50 μm-200 μm in diameter) which gel within five minutes.

FIGS. 3A and 3B show confocal imaging of successfully encapsulated S. meliloti Rm1021 strain. Encapsulated S. meliloti Rm1021 strain with constitutively expressed Green Fluorescent Protein (GFP) within a sample of silica gel. Bacterial distributions within 65 μm×65 m×60 μm 3D projection of bulk silica sample (FIG. 3A) and evenly-spaced z-slices from a 350 m×350 μm×150 μm bulk silica sample (FIG. 3B) to showcase uneven distribution of bacteria within silica matrix. Scale bar indicates 50 m.

FIG. 4 shows in vitro measurement of bacteria holding capacity on alfalfa seeds. Results indicated significantly enhanced bacterial viability with silica gel barrier (GEL). For the alfalfa seeds, the liquid culture bacterization (BAC) had 1,300±800 CFU/seed (n=6) and the silica gel carrier had 10,000±7,000 CFU/seed (n=9). The large increase in viability was likely due to the immobilization of bacteria within the silica gel carrier, allowing the seed to hold additional bacteria. In vitro release was determined by applying inoculants to alfalfa seeds, drying overnight, shaking vigorously for 12 hours, and measuring bacteria released via the plate count technique. A logarithmic scale is used for the y-axis. Error bars indicate standard deviation and square data point indicates mean. A two-sample t-test was used to compare conditions, and one asterisk denotes 0.01<P<0.05.

FIG. 5 shows confocal imaging of an active S. meliloti Rm1021 nodule attached to the root system of a M. sativa plant inoculated with the silica gel carrier. A cross-section of a 1 mm long nodule is displayed with bacteria within the protective encasing of the root nodule.

FIG. 6 shows results demonstrating that Medicago sativa plants inoculated with the silica gel carrier exhibited a significant difference in shoot length from plants deprived of nitrogen sources. Plants inoculated with the silica gel carrier had 84% longer shoots than plants deprived of nitrogen sources. The bacterized plants, which were soaked in raw liquid culture, exhibited similar nodulation and shoot length to plants provided with the rinsed silica gel carrier. At the termination of the six-week greenhouse experiment, the shoot lengths of the nitrogen-deprived control plants (—N) averaged 4.6±0.6 cm, the bacterized plants (BAC) averaged 8±2 cm, and the plants with the silica gel carrier (GEL) averaged 8±2 cm. Error bars indicate standard deviation and connected square data points indicate average height over time.

FIG. 7 shows results demonstrating that Medicago sativa plants inoculated with the rinsed silica gel carrier exhibited a significant difference in dry mass from plants deprived of nitrogen sources. Plants inoculated with the silica gel carrier had 140% more dry mass than plants deprived of nitrogen sources. The bacterized plants, which were soaked in raw liquid culture, exhibited similar nodulation and dry mass to plants provided with the rinsed silica gel carrier. The average dry mass was 9±2 mg (n=12) for the nitrogen-deprived control plants (—N), 18±9 mg (n=21) for the bacterized plants (BAC), and 21±7 mg (n=25) for the plants with the silica gel carrier (GEL). Plants and roots were dried at 60° C. overnight before being weighed individually. Error bars indicate standard deviation and square data point indicates mean. A two-sample t-test was used to compare conditions, and four asterisks denotes P<0.0001 and “ns” denotes no significance (P>0.05).

FIG. 8 shows optical images of two M. sativa seeds coated with silica gel carrier. The nonuniform coating is a result of consecutive spraying of the microbead droplets from an atomizer. A 10× objective lens was used to image the seed surfaces.

FIG. 9 shows in vitro measurement of inoculant holding capacity. Results indicated significantly enhanced bacterial viability when the gel carrier was rinsed. The liquid culture inoculant exhibited 6±3 CFU/bead (n=12), the unrinsed silica gel carrier had 0 CFU/bead (n=16), and the rinsed silica gel carrier had 400±300 CFU/bead (n=19). In vitro release was determined by applying inoculants to 1 mm glass beads, drying overnight, shaking vigorously for 12 hours, and measuring bacteria released via the plate count technique. A logarithmic scale is used for the y-axis. Error bars indicate standard deviation and square data point indicates mean. A two-sample t-test was used to compare conditions, and four asterisks indicate P<0.0001.

FIG. 10 shows total bacteria release from inoculant carriers. Results revealed that the silica gel (GEL) exhibited increased release of bacteria when compared to liquid bacteria culture (BAC). The total release was 200±10 CFU/bead for the silica gel and 5.4±0.6 CFU/bead for the liquid culture during the 144-hr release period. However, bacteria released as a percent of the total bacteria released is approximately the same for both inoculants. Total bacteria release on 1 mm glass beads coated with silica gel microbeads and soaked in liquid bacteria culture. Total release and holding capacity measurements were extrapolated from the bacterial release measurements by summing the measured release over time. Bacterial density refers to colony forming units (CFU) per gram of beads and error bars indicate standard error.

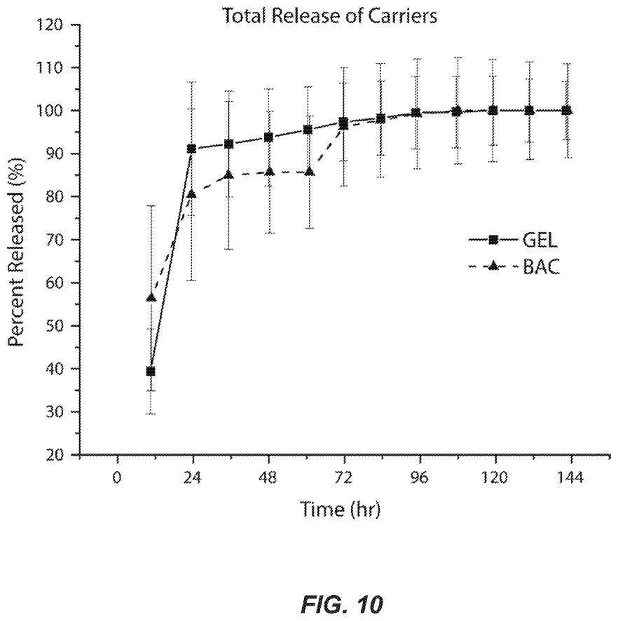

FIG. 11 shows results demonstrating that M. sativa plants inoculated with the silica gel carrier exhibited no significant difference in shoot length from plants deprived of nitrogen sources. The bacterized plants, which were soaked in liquid bacteria culture, exhibited nodulation and increased shoot length when compared to plants deprived of nitrogen sources. At the termination of the six-week greenhouse experiment, the shoot lengths of the nitrogen-deprived control plants (—N) averaged 2.7±0.1 cm, the bacterized plants (BAC) averaged 9±1 cm, and the plants with the silica gel carrier (GEL) averaged 2.9±0.2 cm. Error bars indicate standard deviation and connected square data points indicate average height over time.

FIG. 12 shows results demonstrating that Medicago sativa plants inoculated with the silica gel carrier exhibited no significant difference in dry mass from plants deprived of nitrogen sources. The bacterized plants, which were soaked in liquid bacteria culture, exhibited nodulation and increased dry mass when compared to plants deprived of nitrogen sources. The average dry mass was 9±2 mg (n=7) for the nitrogen-deprived control plants (—N), 30±20 mg (n=17) for the bacterized plants (BAC), and 7±3 mg (n=27) for the plants with the silica gel carrier (GEL). Plants and roots were dried at 60° C. overnight before being weighed individually. Error bars indicate standard deviation and square data point indicates mean. A two-sample t-test was used to compare conditions, and three asterisks indicates 0.0001<P<0.001 and “ns” denotes no significance (P>0.05).

### DETAILED DESCRIPTION

The present disclosure is based, at least in part, on the development of a silica gel-based bacteria carrier and methods for use in encapsulating plant-growth promoting bacteria. Accordingly, aspects of the disclosure are directed to silica gel microbeads comprising one or more plant growth-promoting bacteria. Also disclosed are methods for generating silica gel microbeads, including generating such microbeads on the surface of a plant seed by providing to the plant seed microdroplets of silica-based solution comprising plant growth-promoting bacteria. Further aspects of the disclosure are directed to methods for promoting plant growth comprising providing a plant seed with a solution comprising sodium silicate, citric acid, colloidal silica, and one or more plant growth-promoting bacteria, allowing the solution to gel to form silica gel microbeads, and rinsing the plant seed. Silica gel microbead compositions are also described herein, along with plant seeds coated with a population of silica gel microbeads.

### I. Plant Growth-Promoting Microorganisms

Certain aspects of the present disclosure are related to compositions comprising one or more plant growth-promoting bacteria and methods of using such compositions for increasing one or more plant growth characteristics in plants. Any growth-promoting microorganisms (PGPM) may be used to increase plant growth characteristics in plants. As described elsewhere herein, such PGPM may be attached to or encapsulated in a silica gel microbead. Advantageously, microorganisms of the present disclosure have one or more plant growth-promoting (PGP) activities that allow plants to grow in harsh environments, such as high salt environments, high or low pH environments, low moisture environments, deserts, arid environments, nitrogen-poor environments, low temperature environments, and high temperature environments. For example, microorganisms of the present disclosure may exhibit characteristics including, without limitation, nitrogen fixation, siderophore production, iron chelation, phosphate solubilization, chitinase production, and cellulase production, that promote plant growth in plants grown under harsh environments or in favorable environments. PGPM of the present disclosure include, without limitation, bacteria, such as actinomycetes, firmicutes, and proteobacteria; archaea; fungi; and yeast. In some aspects, PGPM of the present disclosure are plant growth-promoting bacteria (PGPB). “Plant growth-promoting bacteria” or “PGPB,” as used herein, describes any bacteria capable of increasing one or more plant growth characteristics in a plant.

Suitable PGPM of the present disclosure include, without limitation, any PGPMs isolated from the plant tissue, seeds, roots, rhizosphere, plant-associated soil samples, and/or surrounding soil samples from indigenous plants that grow in harsh environmental conditions, such as deserts, arid environments, nitrogen-poor environments, nutrient-poor environments, low pH environments, high pH environments, low temperature environments, and high temperature environments, or from plants that grow in favorable environments. In some aspects, PGPM of the present disclosure exhibit one or more characteristics that include, without limitation, nitrogen fixation, siderophore production, iron chelation, phosphate solubilization, chitinase production, and cellulase production.

In certain aspects, PGPM of the present disclosure include, without limitation, any PGPM from the plant tissue, seeds, roots, rhizosphere, plant-associated soil samples, and/or surrounding soil samples from Vigna plants, including Vigna unguiculata. In certain aspects, PGPM of the present disclosure include, without limitation, any PGPM from the plant tissue, seeds, roots, rhizosphere, plant-associated soil samples, and/or surrounding soil samples from Medicago sativa. In some aspects, the PGPM are isolated from root nodules of Vigna plants. In some aspects, the PGPM are isolated from surface-sterilized root nodules of Vigna plants. In some aspects, the PGPM are isolated using bacterial culture-dependent methods, including trap experiments.

Examples of suitable PGPM of the present disclosure include, without limitation, those listed in Table 1 and Table 2, below.

TABLE 1
Plant growth promoting bacteria

| Strain Designation | NRRL Accession No. |
|---|---|
| Shinella yambaruensis Bots300 | |
| Methylobacterium dankookense Bots301 | B-67981 |
| Methylorubrum populi Bots302 | |
| Methylobacterium radiotolerans Bots303 | B-67982 |
| Sphingomonas leidyi Bots304 | |
| Novosphingobium sp. Bots305 | |
| Lysobacter oryzae Bots306 | |
| Microbacterium lacusdiani Bots307 | |
| Bacillus nealsonii Bots308 | |
| Fictibacillus phosphorivorans Bots309 | B-67983 |
| Bradyrhizobium jicamae Bots310 | |
| Bradyrhizobium sp. Bots311 | |
| Rhizobium milunoense Bots312 | |
| Rhizobium pusense Bots313 | |
| Rhizobium tropici Bots314 | |
| Variovorax sp. 2u118 | |
| Variovorax paradoxus EPS | |
| Variovorax boronicumulans EBFNA2 | |
| Ochrobactrum sp. 1u19 | |
| Ochrobactrum sp. 2u13 | |
| Ochrobactrum sp. 2u114 | |
| Ochrobactrum sp. 2u24 | |
| Bacillus sp. 1u117 | |
| Bacillus sp. PSB43′ | B-67416 |
| Bacillus sp. 1SD10 | |
| Bacillus sp. PSB33 | |
| Bacillus sp. PSB32 | |
| Bacillus sp. PSCA15 | |
| Bacillus sp. 15Sd13 | |
| Bacillus sp. USAFON2 | |
| Bacillus sp. 1SB6 | |
| Bacillus sp. 1SA(ca)5 | |
| Bacillus sp. 1SD11 | |
| Bacillus sp. 1SB5 | |
| Bacillus sp. PSCA21 | |
| Bacillus sp. USAFOC6 | |
| Bacillus sp. USAFONa16 | |
| Oceanobacillus sp. UTRUM2 | |
| Paenibacillus sp. USAFONa6 | |
| Micromonospora sp. USAFONa4 | |
| Micromonospora sp. UTRUM1 | B-67418 |
| Pseudonocardia sp. 2u210 | |
| Streptomyces sp. USAFOC17 | |
| Streptomyces sp. USAFOC20 | |
| Ensifer sp. 1u10 | |
| Ensifer sp. 1u111 | |
| Ensifer sp. 1u113 | |
| Ensifer sp. 1u114 | |
| Ensifer sp. 1u115 | |
| Ensifer sp. 1u116 | |
| Ensifer sp. 2u110 | |
| Ensifer sp. 2u15 | |
| Ensifer sp. 2u16 | |
| Ensifer sp. 2u17 | |
| Ensifer sp. 2u18 | |
| Ensifer sp. 2u27 | |
| Ensifer sp. 4650D | |
| Ensifer sp. 4650F | |
| Ensifer sp. 4677A | |
| Ensifer sp. USAF16 | |
| Ensifer sp. USAF17 | |
| Ensifer sp. 2S(ca)3 | |
| Ensifer sp. PSB71 | |
| Ensifer sp. USAF6 | |
| Ensifer sp. 1u118 | |
| Ensifer sp. USAF 1 | |
| Ensifer sp. USAFON 1 | |
| Rhizobium sp. 1u112a | |
| 1SB12 | |
| 1SB7 | |
| 1SD9 | |
| 1u24b | |
| 2S(Ca)4 | |
| 2S4 | |
| 2u111 | |
| 2u112 | |
| PSB30 | |
| PSB36 | |
| PSB43 | |
| PSB72 | |
| PSB74 | |
| PSCa18 | |
| PSCa25 | |
| PSCA26 | |
| PSCa3 | |
| USAF29PDA | |
| USAFOC | |
| USAFOC8 | |
| USAFON3 | |
| Ornithinibacillus sp. utrum1′ | |
| Paenibacillus pabuli 151 | B-67417 |
| Dietzia cinnamea 55 | B-67422 |
| Lysinobacillus sphaericus 47 | B-67423 |
| Paenibacillus MBEV37 B17 | B-67419 |
| Exiguobacterium alkaliphilum 20 | B-67425 |
| Paenibacillus tundrae 47′ | B-67420 |
| Bacillus simplex 237 | B-67421 |
| B. simplex 30N-2 | |
| Bacillus safensis 34 | B-67620 |
| Bacillus safensis subsp. safensis FO-36b′ | |
| B. subtilis 30VD-1 | |
| Methylobacterium oryzae EBF6NA2 | |

TABLE 2
Plant growth promoting bacteria

Agrobacterium radiobacter
Azospirillum brasilense
Azospirillum lipoferum
Azotobacter chroococcum
Bacillus fimus
Bacillus licheniformis
Bacillus megaterium
Bacillus mucilaginous
Bacillus pumilus
Bacillus spp.
Bacillus subtilis
Bacillus subtilis var. amyloliquefaciens
Bradyrhizobium japonicum USDA 110
Burkholderia cepacia
Delfitia acidovorans
Paenobacillus macerans
Pantoea agglomerans
Pseudomonas aureofaciens
Pseudomonas chlororaphis
Pseudomonas fluorescens
Pseudomonas solanacearum
Pseudomonas spp.
Pseudomonas syringae
Serratia entomophilia
Sinorhizobium meliloti Rm1021
Streptomyces griseoviridis
Streptomyces spp.
Streptomyces lydicus

In some aspects, PGPM of the present disclosure also include homologues, variants, and mutants of the PGPM listed in Table 1 or Table 2. In some aspects, the homologues, variants, and mutants of the PGPM listed in Table 1 and Table 2 have all the identifying characteristics of the PGPM listed in Table 1 and Table 2.

Additional PGPM and PGPB are known in the art and contemplated herein. Details regarding certain PGPM are described in, for example, U.S. Pat. Nos. 10,555,532, 10,212,943, PCT Patent Publication WO/2020/018694, and U.S. Provisional Application 63/072,413, and Glick B. R. (2012). Plant growth-promoting bacteria: mechanisms and applications. Scientifica, 2012, 963401, all of which are incorporated herein by reference in their entirety.

### II. Plant Growth-Promoting Compositions

Other aspects of the present disclosure relate to plant growth-promoting (PGP) compositions containing one or more PGPB of the present disclosure for increasing one or more plant growth characteristics in plants. For example, as disclosed herein, a PGP composition of the disclosure may be a silica-based composition (e.g., silica solutions, silica microbeads, etc.) comprising one or more PGPB. In some aspects, a PGP composition of the disclosure is provided to a plant seed. In some aspects, the PGP composition may include from one or more to 15 or more PGPB. In other aspects, the PGP composition includes one or more, two or more, three or more, four or more, five or more, six or more, seven or more, eight or more, nine or more, 10 or more, or 15 or more PGPB.

In order to achieve an increase in one or more plant growth characteristics, or to improve upon an increase in one or more plant growth characteristics, the PGP compositions of the present disclosure may also include other components or mixture of components to facilitate the viability of the PGPB; inoculation of the plant, plant parts thereof, or rhizospheres; or transportation or storage of the compositions. Any suitable components known in the art may be used.

In some aspects, the PGP compositions of the present disclosure may further contain a carrier for delivering, inoculating, or otherwise growing a plant in the presence of the composition in order to promote plant growth and productivity, such as germination, yield, and the like, by increasing one or more plant growth characteristics. Any suitable carrier known in the art may be used, including without limitation, a liquid, a solid, and a combination of a liquid and a solid carrier. In some aspects, the liquid carrier may include water.

PGP compositions of the present disclosure may further contain components for providing additional benefits to the PGPB or plants, including without limitation, an herbicide, a pesticide, a fungicide, a plant growth regulator, and an encapsulation agent, a wetting agent, a dispersing agent, and the like for enhancing the effect of the PGP composition. One or more of these components may be excluded from a PGP composition of the disclosure in certain aspects.

### A. Silica-Based Solutions and Compositions Comprising Plant Growth-Promoting Bacteria

In some aspects, a plant growth-promoting (PGP) composition of the disclosure is a silica-based composition comprising one or more PGPB. Silica-based compositions include, for example, silica-containing solutions, gels, and solids. In some aspects, a silica-based composition of the disclosure is a solution comprising sodium silicate, citric acid, colloidal silica, and one or more PGPB (described herein in certain aspects as a “bacteria-containing silica-based solution”). Such a solution may be formed by, for example, mixing a silica solution comprising sodium silicate, citric acid, and colloidal silica and a bacteria solution comprising one or more PGPB.

In some aspects, a silica-based solution is for use in generation of droplets (e.g., microdroplets), where such droplets gel to form silica gel microbeads (also “silica microbeads” or “silica sol-gel microbeads”). For example, a silica-based solution may be applied as droplets to a plant seed. In such cases, it may be desirable to formulate a silica-based solution such that the gelation process occurs slowly enough to provide sufficient time for such droplet formation and application. Accordingly, aspects of the present disclosure are directed to silica-based solutions (e.g., silica-based solutions comprising one or more PGPB) that gel only after at least 1, 2, 3, 4, or 5 minutes (or any range or value therein) following formation of the solution. In some aspects, a silica-based solution of the disclosure does not gel until at least 1, 2, 3, 4, or 5 minutes (or any range or value therein) following formation of the solution. In some aspects, a silica-based solution of the disclosure does not gel less than 1, 2, 3, 4, or 5 minutes (or any range or value therein) following formation of the solution. In some aspects, the solution does not gel less than 2 minutes following formation of the solution. In some aspects, the solution does not gel less than 3 minutes following formation of the solution. In some aspects, the solution does not gel less than 4 minutes following formation of the solution.

In some aspects, a silica-based solution of the disclosure (e.g., a bacteria-containing silica-based solution) comprises a silicate salt. In some aspects, the silicate salt is sodium silicate. In some aspects, a silica-based solution comprises at least, at most, or about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mol/L sodium silicate, or any range or value derivable therein. In some aspects, the solution comprises between 1.0 and 2.0 mol/L sodium silicate. In some aspects, the solution comprises between 1.4 and 1.6 mol/L sodium silicate. In some aspects, the solution comprises about 1.5 mol/L sodium silicate. In some aspects, the solution comprises 1.5 mol/L sodium silicate.

In some aspects, a silica-based solution of the disclosure (e.g., a bacteria-containing silica-based solution) comprises citric acid. In some aspects, a silica-based solution comprises at least, at most, or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 mol/L citric acid, or any range or value derivable therein. In some aspects, the solution comprises between 0.5 and 1.5 mol/L citric acid. In some aspects, the solution comprises between 0.9 and 1.1 mol/L citric acid. In some aspects, the solution comprises about 1.0 mol/L citric acid. In some aspects, the solution comprises 1.0 mol/L citric acid.

In some aspects, a silica-based solution of the disclosure (e.g., a bacteria-containing silica-based solution) comprises colloidal silica. In some aspects, the solution comprises a ratio of the colloidal silica to the sodium silicate of between 2:4 and 4:4. In some aspects, the solution comprises a ratio of the colloidal silica to the sodium silicate of about 3:4. In some aspects, the solution comprises a ratio of the colloidal silica to the sodium silicate of 3:4.

In some aspects, a silica-based solution of the disclosure (e.g., a bacteria-containing silica-based solution) has a pH of at least, at most, or about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, or any range or value derivable therein. In some aspects, the solution has a pH of between 6.5 and 7.5. In some aspects, the solution has a pH of between 6.9 and 7.1. In some aspects, the solution has a pH of about 7.0. In some aspects, the solution has a pH of 7.0.

In some aspects, a bacteria-containing silica-based solution of the disclosure comprises 1, 2, 3, 4, 5, 6, 7, 8, 9, 10, or more PGPB. In some aspects, the solution comprises at least, at most, or about 10⁴, 10⁵, 10⁶, 10⁷, 10⁸, or 10⁹ CFU/mL of PGPB. In some aspects, the solution comprises between 10⁵ and 10⁸ CFU/mL of PGPB, or any range or value derivable therein. In some aspects, the solution comprises about 10⁷ CFU/mL of PGPB. In some aspects, the solution comprises 10⁷ CFU/mL of PGPB.

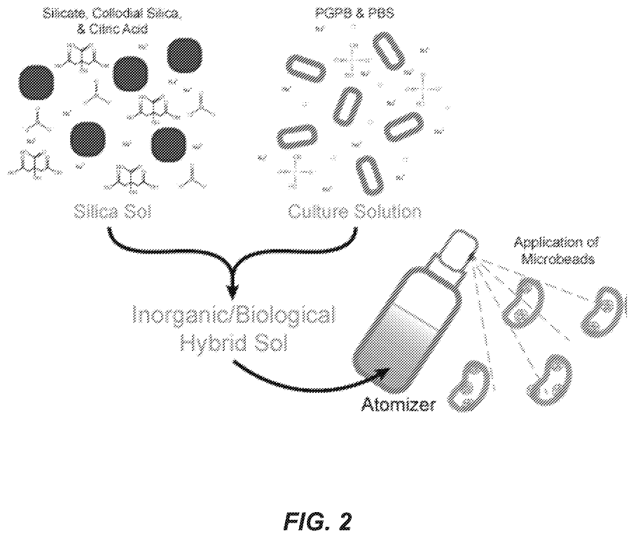

In some aspects, a PGP composition of the disclosure is a silica gel microbead comprising (e.g., encapsulating, coated with, etc.) one or more PGPB (described herein in some aspects as a “bacteria-containing silica gel microbead”). A silica gel microbead may be produced from a silica-based solution using a sol-gel synthesis method. In some aspects, a bacteria-containing silica gel microbead is produced by generating a solution comprising sodium silicate, citric acid, colloidal silica, and one or more plant growth-promoting bacteria, followed by generating droplets (e.g., microdroplets) from the solution. Droplets may be generated by various methods including, for example, spraying the solution with an atomizer. Following formation of droplets, the droplets may be allowed to gel to form silica gel microbeads. As disclosed herein, silica gel microbeads may be formed on the surface of a plant seed in some aspects. An example of such a process is shown in FIG. 2.

In some aspects, a silica-based solution droplet of the disclosure has a diameter of at least, at most, or about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 μm, or any range or value derivable therein. A droplet may comprise at least, at most, or about 5, 6, 7, 8, 9, 10, 11, 12, 13, 14, 15, 16, 17, 18, 19, 20, 21, 22, 23, 24, 25, 26, 27, 28, 29, or 30% silica by weight, or any range or value derivable therein. In some aspects, a droplet comprises between 10% and 20% silica by weight. In some aspects, a droplet comprises about 13% silica by weight. In some aspects, a droplet comprises 13% silica by weight.

In some aspects, a silica gel microbead of the disclosure has a diameter of at least, at most, or about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 μm, or any range or value derivable therein. In some aspects, a silica gel microbead has a diameter of between about 50 μm and about 200 μm. In some aspects, a silica gel microbead has a diameter of between 50 μm and 200 μm.

### III. Plants

Other aspects of the present disclosure relate to growing plants in the presence of a PGP composition in order to increase one or more plant growth characteristics in the plant. In some aspects, growing a plant in the presence of a PGP composition comprises coating a plant seed in a PGP composition. For example, in some aspects, disclosed is a method comprising providing to a plant seed a silica-based solution comprising PGPB. In some aspects, the solution comprises sodium silicate, citric acid, colloidal silica, and one or more PGPB. A plant seed may be coated in droplets of the silica-based solution, which, following gelling of the solution, results in a plant seed coated in silica microbeads comprising the one or more PGPB. In some aspects, growing such a coated plant seed results in improvement of one or more plant growth characteristics compared to a plant seed not provided any PGPB.

Plants of the present disclosure may be of any kind or from any source known in the art. For example, suitable plants of the present disclosure include, without limitation, those intended to be grown in harsh environments, such as plants grown in soils that are dry, acidic, or both; plants that are prone to infection by pathogens, such as fungi; plants grown in a desert or arid environment; plants grown in nitrogen-poor environments; plants grown in nutrient-poor environments; plants grown in low pH conditions; plants grown in high pH conditions; plants grown in low temperature conditions; and plants grown in high temperature conditions. Suitable plants of the present disclosure may be native to such harsh environments, or may plants grown in harsh environments but that are not native to such harsh environments. Suitable plants used with the compositions and methods of the present disclosure may be grown in any environment or in any growth medium, such as solid medium or liquid medium. Suitable plants of the present disclosure may also include plants that are grown in favorable conditions.

Suitable plants of the present disclosure include, without limitation, crop plants, energy crop plants, plants that are used in agriculture, and plants used in industrial settings. Plants of the present disclosure may be either monocotyledons or dicotyledons. For example, suitable plants of the present disclosure include, without limitation, desert plants, desert perennials, legumes, such as Medicago sativa (alfalfa), Lotus japonicus, Melilotus alba (sweet clover), Pisum sativum (pea), and Vigna unguiculata (cowpea), Mimosa pudica, Lupinus succulentus (lupine), Macroptilium atropurpureum (siratro), Medicago truncatula, and Trifolium repens (white clover), corn, sorghum, miscanthus, sugarcane, poplar, spruce, pine, wheat, rice, soy, cotton, barley, turf grass, tobacco, potato, bamboo, rape, sugar beet, sunflower, willow, eucalyptus, Amorphophallus spp., Amorphophallus konjac, giant reed (Arundo donax), reed canarygrass (Phalaris arundinacea), Miscanthus giganteus, Miscanthus sp., sericea lespedeza (Lespedeza cuneata), millet, ryegrass (Lolium multiflorum, Lolium sp.), timothy, Kochia (Kochia scoparia), forage soybeans, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, and Kentucky bluegrass.

In certain aspects, the plants are dicotyledons. It will be apparent to one of skill in the art that the plants of the present disclosure may also include nodulating plants. In other aspects, the plants are desert plants, desert perennials, crop plants, or legumes. In certain aspects, the plant are legumes, including without limitation, Medicago sativa, (alfalfa), Lotus japonicus, Melilotus alba (sweet clover), Pisum sativum (pea), and Vigna unguiculata (cowpea), Mimosa pudica, Lupinus succulentus (lupine), Macroptilium atropurpureum (siratro), Medicago truncatula and Trifolium repens (white clover).

In some aspects, the plant is Medicago sativa (alfalfa).

### IV. Plant Growth Characteristics

In some aspects, PGPM (e.g., PGPB such as PGPB encapsulated in silica gel microbeads) of the present disclosure increase one or more plant growth characteristics of plants of the present disclosure. Plant growth characteristics of the present disclosure include, without limitation, plant biomass, plant growth rate, plant yield, root biomass, nodulation, nitrogen utilization, nutrient utilization, salt tolerance, resistance to one or more pathogens, resistance to fungal growth, growth under arid conditions, growth under arid soil conditions, growth under low pH conditions, growth under low pH soil conditions, growth under high pH conditions, growth under high pH soil conditions, growth under low temperature conditions, growth under low temperature soil conditions, growth under high temperature conditions, and growth under high temperature soil conditions. As will be apparent to one of skill in the art, certain characteristics, for example nodulation, include other forms of life that interact with the plant.

As used herein, “increasing one or more plant growth characteristics” refers to increasing, without limitation, plant biomass, plant growth rate, plant yield, root biomass, nodulation, nitrogen utilization, nutrient utilization, salt tolerance, resistance to one or more pathogens, resistance to fungal growth, growth under arid conditions, growth under arid soil conditions, growth under low pH conditions, growth under low pH soil conditions, growth under high pH conditions, growth under high pH soil conditions, growth under low temperature conditions, growth under low temperature soil conditions, growth under high temperature conditions, and growth under high temperature soil conditions of a plant grown in the presence of a PGPB of the present disclosure, as compared to a corresponding plant grown under the same conditions but in the absence of the PGPB.

In certain aspects, growing a plant in the presence of one or more PGPB of the present disclosure increases, without limitation, plant biomass, plant growth rate, plant yield, root biomass, nodulation, nitrogen utilization, nutrient utilization, salt tolerance, resistance to one or more pathogens, resistance to fungal growth, growth under arid conditions, growth under arid soil conditions, growth under low pH conditions, growth under low pH soil conditions, growth under high pH conditions, growth under high pH soil conditions, growth under low temperature conditions, growth under low temperature soil conditions, growth under high temperature conditions, and/or growth under high temperature soil conditions by about 5% to about 200%, or any range or value derivable therein, as compared to a corresponding plant grown under the same conditions but in the absence of the one or more PGPB of the present disclosure. In some aspects, growing a plant in the presence of one or more PGPB of the present disclosure increases, without limitation, plant biomass, plant growth rate, plant yield, root biomass, nodulation, nitrogen utilization, nutrient utilization, salt tolerance, resistance to one or more pathogens, resistance to fungal growth, growth under arid conditions, growth under arid soil conditions, growth under low pH conditions, growth under low pH soil conditions, growth under high pH conditions, growth under high pH soil conditions, growth under low temperature conditions, growth under low temperature soil conditions, growth under high temperature conditions, and/or growth under high temperature soil conditions by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, or about 200%, or by a range between any two of these values, as compared to a corresponding plant grown under the same conditions but in the absence of the one or more PGPB of the present disclosure.

In other aspects, growing a plant in the presence of one or more PGPB of the present disclosure increases, without limitation, plant biomass, plant growth rate, plant yield, root biomass, nodulation, nitrogen utilization, nutrient utilization, salt tolerance, resistance to one or more pathogens, resistance to fungal growth, growth under arid conditions, growth under arid soil conditions, growth under low pH conditions, growth under low pH soil conditions, growth under high pH conditions, growth under high pH soil conditions, growth under low temperature conditions, growth under low temperature soil conditions, growth under high temperature conditions, and/or growth under high temperature soil conditions by about 2-fold to about 100-fold, or any range or value derivable therein, as compared to a corresponding plant grown under the same conditions but in the absence of the one or more PGPB of the present disclosure. In some aspects, growing a plant in the presence of one or more PGPB of the present disclosure increases, without limitation, plant biomass, plant growth rate, plant yield, root biomass, nodulation, nitrogen utilization, nutrient utilization, salt tolerance, resistance to one or more pathogens, resistance to fungal growth, growth under arid conditions, growth under arid soil conditions, growth under low pH conditions, growth under low pH soil conditions, growth under high pH conditions, growth under high pH soil conditions, growth under low temperature conditions, growth under low temperature soil conditions, growth under high temperature conditions, and/or growth under high temperature soil conditions by about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, or about 100-fold, or by an amount between any two of these values, as compared to a corresponding plant grown under the same conditions but in the absence of the one or more PGPB of the present disclosure.

As disclosed herein, plant biomass and yield refer to the accumulation of plant matter in any part or all of the plant, with yield including, without limitation, the crop production of crop plants.

As disclosed herein, nodulation includes any process or quality associated with root nodule formation, including but not limited to nodule size, color, clustering, development, branching of vascular bundles, and colonization by rhizobia.

As disclosed herein, nitrogen and nutrient utilization include, without limitation, how well nitrogen or nutrients are taken up by the plant, the amounts of nitrogen or nutrients present in the plant, tissues thereof, or surrounding soil environment, and/or how efficiently the nitrogen or nutrients are incorporated or utilized by the plant.

As disclosed herein, resistance to pathogens or fungal growth includes, without limitation, increased plant survival upon infection with pathogen or fungal growth, a decreased growth rate or size of pathogen or fungal growth on or near the plant, or a diminished frequency with which pathogen or fungal growth appears on or near the plant.

As disclosed herein, arid conditions and arid soil conditions refer to any environment in which the plant and its immediate surroundings receive less than 50 mm of water per month. Arid conditions and arid soil conditions may also refer to any environment characterized by irregular exposure of plants to water, regardless of the total amount received.

As disclosed herein, low pH conditions and low pH soil conditions refer to any environment for plant growth with a pH of between about 0.0 to about 6.0, for example about 0.0, about 0.5, about 1.0, about 1.5, about 2.0, about 2.5, about 3.0, about 3.5, about 4.0, about 4.5, about 5.0, about 5.1, about 5.2, about 5.3, about 5.4, about 5.5, about 5.6, about 5.7, about 5.8, about 5.9, or about 6.0, or below any of these values, or between any two of these values. High pH conditions and high pH soil conditions refer to any environment for plant growth with a pH of about 6.1 to about 14, for example about 6.1, about 6.2, about 6.3, about 6.4, about 6.5, about 6.7, about 6.8, about 6.9, about 7.0, about 7.5, about 8.0, about 8.5, about 9.0, about 9.5, about 10, about 10.5, about 11, about 11.5, about 12, about 12.5, about 13, about 13.5, or about 14, or above any of these values, or between any two of these values.

As disclosed herein, low temperature and low temperature soil conditions refer to an ambient or soil temperature less than or equal to 15° C., for example less than or equal to −30° C., less than or equal to −25° C., less than or equal to −20° C., less than or equal to −15° C., less than or equal to −10° C., less than or equal to −9° C., less than or equal to −8° C., less than or equal to −7° C., less than or equal to −6° C., less than or equal to −5° C., less than or equal to −4° C., less than or equal to −3° C., less than or equal to −2° C., less than or equal to −1° C., less than or equal to −0° C., less than or equal to 1° C., less than or equal to 2° C., less than or equal to 3° C., less than or equal to 4° C., less than or equal to 5° C., less than or equal to 6° C., less than or equal to 7° C., less than or equal to 8° C., less than or equal to 9° C., less than or equal to 10° C., less than or equal to 11° C., less than or equal to 12° C., less than or equal to 13° C., less than or equal to 14° C., or less than or equal to 15° C., or less than or equal to any of these values, or between any two of these values. High temperature and high temperature soil conditions refer to an ambient or soil temperature greater than or equal to 50° C., for example greater than or equal to 15° C., greater than or equal to 20° C., greater than or equal to 25° C., greater than or equal to 26° C., greater than or equal to 27° C., greater than or equal to 28° C., greater than or equal to 29° C., greater than or equal to 30° C., greater than or equal to 31° C., greater than or equal to 32° C., greater than or equal to 33° C., greater than or equal to 34° C., greater than or equal to 34° C., greater than or equal to 35° C., greater than or equal to 36° C., greater than or equal to 37° C., greater than or equal to 38° C., greater than or equal to 39° C., greater than or equal to 40° C., greater than or equal to 41° C., greater than or equal to 42° C., greater than or equal to 43° C., greater than or equal to 44° C., greater than or equal to 45° C., greater than or equal to 46° C., greater than or equal to 47° C., greater than or equal to 48° C., greater than or equal to 49° C., or greater than or equal to 50° C., or greater than any of these values or between any two of these values.

### V. Contacting and Growing Plants with Plant Growth-Promoting Compositions

Aspects of the disclosure are directed to plant seeds coated with a population of silica gel microbeads, and methods for making and using such coated plant seeds. A plant seed disclosed herein may be described as “coated” with a population of silica gel microbeads. As used herein, a plant seed “coated” with a population of silica gel microbeads describes a plant seed having, attached to its outer surface, a population of two or more silica gel microbeads. A plant seed may be coated with a population of silica gel microbeads comprising, comprising at least, or comprising at most 1, 2, 3, 4, 5, 6, 7, 8, 9, 10, 20, 30, 40, 50, 100, 200, 300, 400, 500, 1000, 5000, or 10000 microbeads (or any range or value derivable therein), or more. A population of silica gel microbeads may comprise one or more PGPB. In such aspects, a plant seed may be coated with a population of silica gel microbeads comprising, comprising at least, or comprising at most 50, 100, 200, 300, 400, 500, 600, 700, 800, 900, 1000, 1500, 2000, 3000, 4000, 5000, 6000, 7000, 8000, 9000, 10000, 15000, 20000, 30000, 40000, or 50000 CFU of PGPB, or any range or value derivable therein.

As disclosed herein, silica gel microbeads may be generated by generation of droplets (e.g., microdroplets) from a silica-based solution, where such droplets gel to form silica gel microbeads. Accordingly, in some aspects, disclosed are methods for coating a plant seed with silica gel microbeads comprising applying silica-based solution droplets to a plant seed and allowing the droplets to gel to form the silica gel microbeads. In some aspects, the silica-based solution is a bacteria-containing silica-based solution. As disclosed herein, in certain aspects it is desirable to reduce the amount of sodium silicate on a plant seed after coating with silica gel microbeads comprising one or more PGPB, for example to improve viability of the PGPB. Accordingly, in some aspects, after allowing the droplets to gel, a plant seed is rinsed. Rinsing a plant seed may comprise application of any solution to the plant seed, thereby reducing the amount of sodium silicate on the plant seed. In some aspects, the plant seed is rinsed with water. In some aspects, the plant seed is rinsed with a buffer solution. In some aspects, the buffer solution is phosphate buffered saline. In some aspects, the buffer solution is tris buffered saline. A plant seed may be rinsed for at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any range or value derivable therein. In some aspects, the plant seed is rinsed for at least 10 minutes. In some aspects, the plant seed is rinsed for at least 15 minutes. In some aspects, the plant seed is rinsed for at least 20 minutes. A plant seed may be rinsed 1, 2, 3, 4, 5, 6, 7, 8, 9, or 10 times, or more. In some aspects, the plant seed is rinsed once. In some aspects, the plant seed is rinsed twice.

In some aspects, plants are grown in the presence of PGP compositions of the present disclosure. Any suitable method known in the art for growing plants in the presence of microorganisms and disclosed herein may be used. As disclosed herein, PGPM may be used in any state or temperature that does not adversely affect the viability. For example, the PGPM may be prepared as liquid cultures, lyophilized powders, air-dried powders, freeze-dried powders, beads, spores, aqueous slurries, gums, encapsulated in silica gel microbeads, or prepared within soil or peat preparations.

In some aspects, growing a plant in the presence of one or more PGPM of the present disclosure includes contacting one or more PGPM of the present disclosure with plant seed. For example, plant seeds may be coated with the one or more PGPM of the present disclosure, in liquid or solid suspensions, directly or in combination with any type of suitable carrier known in the art, including without limitation, any medium, suspension, powder, clay, oil, peat, and the like. Alternatively, the one or more PGPM of the present disclosure may be absorbed into a granular carrier (e.g., pelleted peat) that is planted with the seed.

In other aspects, growing a plant in the presence of one or more PGPM of the present disclosure includes contacting one or more PGPM of the present disclosure with a plant or part thereof. For example, the one or more PGPM of the present disclosure may be added to any part of the plant, including without limitation, stems, flowers, leaves, nodes, aerial roots, and underground roots, using any suitable method known in the art. The one or more PGPM of the present disclosure may be added at any time during plant growth, or in combination with any other treatment, for example, with fertilizers, pesticides, fungicides, or any combination thereof.

In further aspects, growing a plant in the presence of one or more PGPM of the present disclosure includes contacting one or more PGPM of the present disclosure with plant roots or the plant rhizosphere. For example, the one or more PGPM of the present disclosure may be encapsulated in beads or in any other carrier and applied to the plant roots or rhizosphere. Alternatively, the one or more PGPM of the present disclosure may be added to the soil or other suitable growth medium containing the rhizosphere using any suitable method known in the art. As used herein, the plant rhizosphere may include, without limitation, roots, root nodules, root caps, root secretions, rhizosphere-associated microorganisms, and rhizosphere-associated soil.

As disclosed herein, the one or more PGPM of the present disclosure may be used at any concentration or dose sufficient to increase one or more plant growth characteristics of a plant that is grown in the presence of such PGPM.

### Examples

The following examples are included to demonstrate certain aspects of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the invention.

### Example 1—Silica Sol-Gel Microbead Encapsulation for Delivery of Symbiotic Microbes

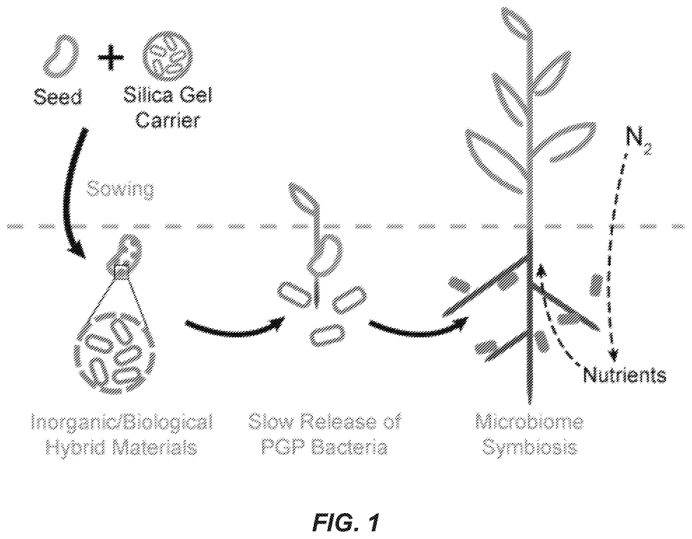

Described is the development of microbe-encapsulating microbeads using an inorganic nanomaterial: silica produced through the sol-gel process (FIG. 1). Silica gels meet many of the desired requirements of microbe carriers, including chemical inertness, biocompatibility, biodegradability, resistance to microbial attack, buffer-holding capacity, and increased mechanical strength compared to organic polymers.⁹⁻¹¹ The sol-gel method is a promising candidate for bacterial inoculation due to the low cost and the availability of precursors, the ability to preserve living cells in highly porous polymers, and the highly controllable gelation of the silica matrix.⁹⁻¹¹ Therefore, due to the increased abilities and strength of inorganic silica gels, it was hypothesized that microbeads made with a silica sol-gel formulation would take longer to degrade than current inoculant carriers, resulting in a sustained release of microbes and increase in plant growth over other PGPB carriers. In this work, a legume-Rhizobium system is studied. Specifically, the Sinorhizobium meliloti Rm1021 strain, a bacterium capable of nitrogen fixation and creating symbiotic nodules on legume plants, was used with Medicago sativa due to their natural symbiotic association.¹³ Confocal microscopy was utilized to examine the distribution of S. meliloti within the carrier. In vitro experimentation allowed for measurement of microbial viability. To measure the effectiveness of the silica gel carrier, plant assays with Medicago sativa were used to determine the resulting plant shoot length and plant dry mass.

### Methods

Bacteria growth. Sinorhizobium meliloti Rm1021 was the PGPB strain used to demonstrate the viability of the silica gel as a carrier. The S. meliloti strain included the plasmid pHC60, which constitutively expressed green fluorescent protein (GFP) and resistance to tetracycline.^{14,15} The strain was cultured at 1.79 relative centrifugal force (RCF) and 30° C. for two days in Tryptone-Yeast extract complex medium (0.5 g/L CaCl₂), 3 g/L yeast extract, 5 g/L tryptone, and adjusted to pH 7.2) prepared with 10 μg/mL tetracycline. Optical density (OD) measurements at a wavelength of 600 nm and the plate count technique were used to determine that the colony forming unit (CFU) density at an OD₆₀₀ of 1.0 was 1×10⁷ CFU/mL.

Preparation of Bacteria-Encapsulated Silica Gel. Bacteria-containing silica hydrogel microbeads were developed by combining silica sol and a bacteria culture solution to create an inorganic and biological hybrid sol that was sprayed to produce microbead droplets (FIG. 2). Through the use of an aqueous pathway using a stoichiometric ratio of 1.5 mol/L sodium silicate (Beantown Chemical) and 1.0 mol/L citric acid (Sigma-Aldrich), the sol-gel process was utilized to produce a homogenous solution that could gel quickly at ambient conditions. LUDOX® HS-40 colloidal silica (Sigma-Aldrich) was combined with the sodium silicate in a 3:4 volume ratio to increase the silica concentration in the final gel without increasing ionic strength to slow the gelation process. 10× concentrated phosphate-buffered saline (10×PBS) of pH 7.4 was added to the citric acid in a 1:8 volume ratio to increase the buffering capacity of the final gel. These solutions were homogenized, mixed with the previously described bacteria culture that was resuspended in 1×PBS of pH 7.2, further homogenized, and passed through an atomizer to produce micrometer-scale droplets (50 μm-200 μm in diameter). The silica gel microbeads gelled at ambient conditions within five minutes and were rinsed twice for 20 minutes with PBS of pH 7.0 prior to overnight drying. The wet hydrogel was approximately pH 7, contained a calculated 13.0 percent silica by weight, and was loaded with approximately 2×10⁷ CFU/mL.

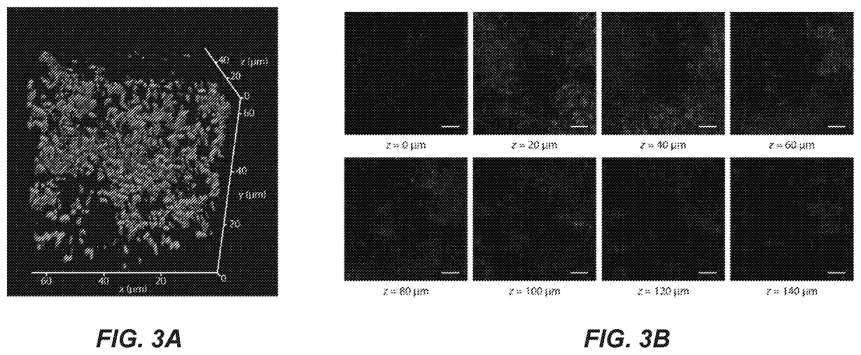

Confocal Microscopy. To observe bacteria encapsulated within the silica gel, bulk samples of gel were prepared on #1.5 coverslips and examined using confocal microscopy. The coverslips were cleaned with oxygen plasma for two minutes and then treated with hexamethyldisilane vapor for 10 minutes. The sample was mounted on the slide holder of an inverted confocal laser scanning microscope (Leica SP8 SMD). A 488 nm laser was used as the excitation light, and the emission between 500 nm-550 nm was collected with a photomultiplier tube detector. For results shown in FIG. 3A, a 100× immersive type objective lens (Leica 100× HC PL APO OIL CS2 NA/1.4) was used for observation, and the scanner worked on x-y-z mode at an x-y spatial resolution of 63 nm/pixel, taking x-y cross-sectional images at a z increment of 1.0 μm from the bottom of the gel. A series of z-stack images were acquired, processed, and reconstructed into three-dimensional images using Leica Application Suite X (LASX). For results shown in FIG. 3B, a 20× immersive type objective lens (Leica 20× HC PL APO IMM CS2 NA/0.75) was used for observation, and the scanner worked on x-y-z mode at an x-y spatial resolution of 342 nm/pixel, taking x-y cross-sectional images at a z increment of 1.0 μm from the bottom of the gel.

Viability of Encapsulated Bacteria. To measure the viability of the bacteria within the silica gel carrier, the prepared Medicago sativa (alfalfa) seeds were shaken at 1.79 RCF in 10 mL PBS of pH 7.0 for 12 hours at 30° C. to remove viable bacteria. The number of bacteria that remained viable (i.e., the bacterial units that formed colonies) was extrapolated using the plate count technique after several days of incubation at 30° C. To compare the viability of bacteria using the silica gel carrier to an existing inoculation method, an equivalent number of seeds were bacterized with liquid bacteria culture. The seeds were shaken in 10 mL of bacteria culture of OD₆₀₀=1.0 for two hours at 0.448 RCF to attach bacteria to the seed surface. Once attached, the seeds were stored overnight and shaken at 1.79 RCF in 10 mL PBS of pH 7.0 for 12 hours at 30° C. to remove viable bacteria, which was measured by the plate count technique and subsequent incubation. A two-sample t-test was used to determine significance between the viable bacteria released by the silica gel carrier and the bacterized seeds.

Greenhouse Plant Assays. Greenhouse studies with Medicago sativa (alfalfa) plants determined the effectiveness of the silica sol-gel microbead carrier against bacterization with liquid culture and a control group deprived of nitrogen. Sterilized alfalfa seeds were coated with the dry silica microbeads before sowing. Other seeds were treated bacterized with liquid bacteria culture. Six seeds per pot were sown 1 cm below the surface in 3.6 L pots containing a 1:2 mixture of perlite (Therm-O-Rock West, Inc.) to vermiculite (Therm-O-Rock West, Inc.). Throughout the six-week experiment, all pots were watered twice-weekly with 200 mL of nitrogen-deficient Hoagland solution,¹⁶ the pot locations within the growing space were randomized weekly, and plant shoot lengths were recorded at least once per week. At the termination of the assay, plants and their roots were removed from the soil, and several indicators of growth were recorded: shoot length, number and activity of nitrogen-fixing nodules, and dry mass of the plants. Dry mass was determined by drying the plants overnight at 60° C. and weighing each plant individually. A two-sample t-test was used to determine the significance of the dry mass results.

### Results

Confocal microscopy of encapsulated S. meliloti. To determine that the bacteria were successfully encapsulated within the silica matrix, confocal laser scanning microscopy was used to examine bulk samples of silica gel (FIGS. 3A and 3B). The S. meliloti Rm1021 was found dispersed within the gel, mostly as isolated bacteria, but also with regions of varying bacterial density. The relative homogeneity within the x-y slices of the gel signified that the simple methodologies employed were sufficient to produce a useful inoculant which, when sprayed to form microbeads, remained relatively uniform in composition.

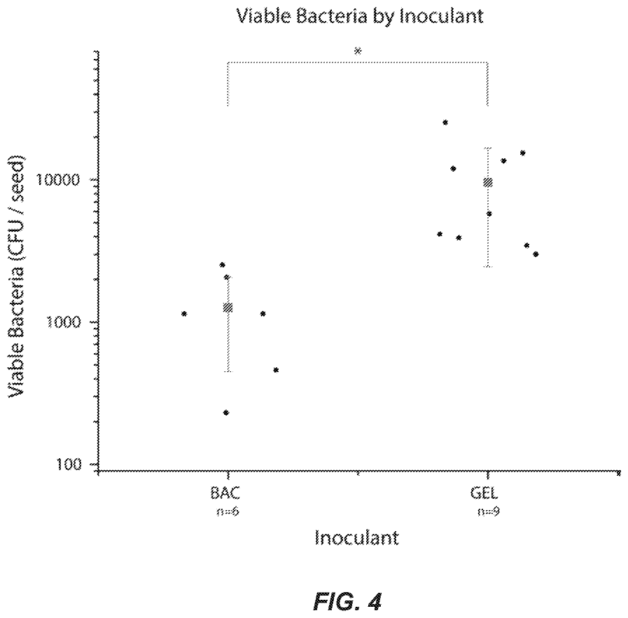

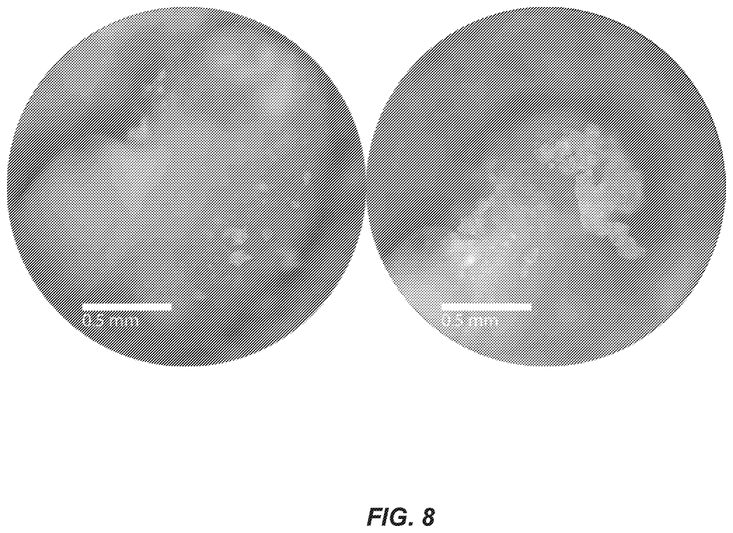

Holding capacity of silica gel carrier. In vitro viability experiments with alfalfa seeds were employed to measure the viability of the bacteria within the silica gel carrier and to compare to seeds bacterized with liquid bacteria culture. For the viability on inoculated alfalfa seeds (FIG. 4), the seeds coated in the rinsed silica gel carrier had an average of 10,000±7,000 CFU/seed, corresponding to a calculated 19% viability of the bacteria in the initial sprayed solution. The seeds coated in liquid culture had an average of 1,300±800 CFU/seed, or approximately one order of magnitude lower than the silica gel carrier. The difference in holding capacity between the inoculation methods is likely attributed to the silica gel carrier, which contains immobilized bacteria, solidifying on the seed surface (FIG. 8), whereas the bacterized seeds require bacteria adhering to the seed directly.

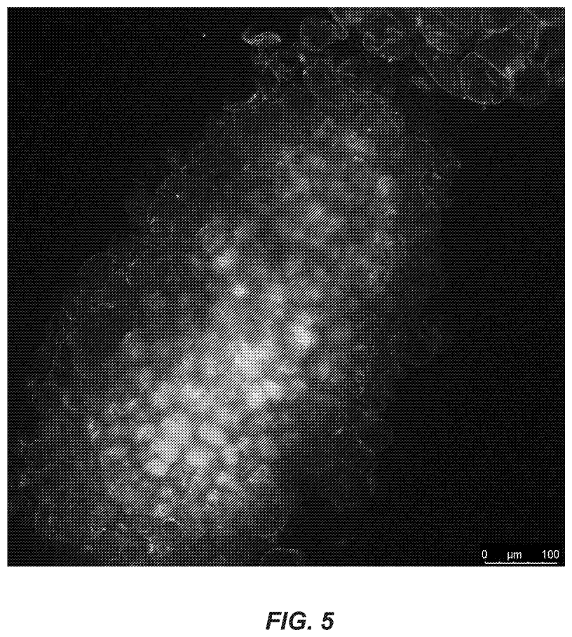

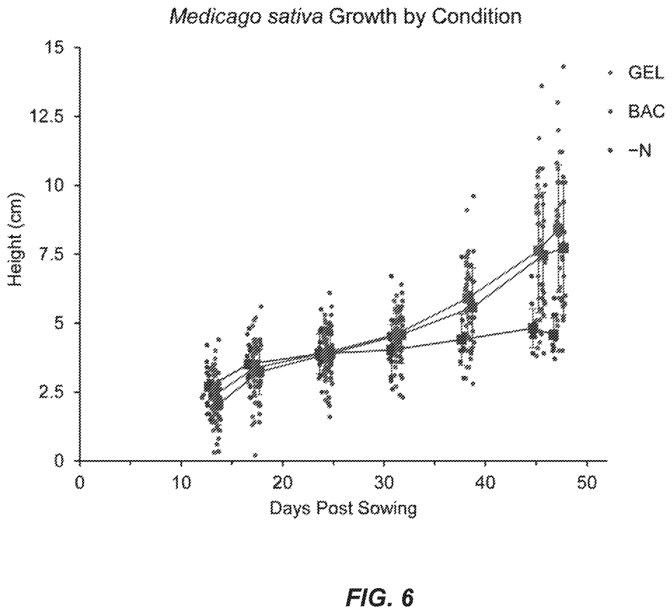

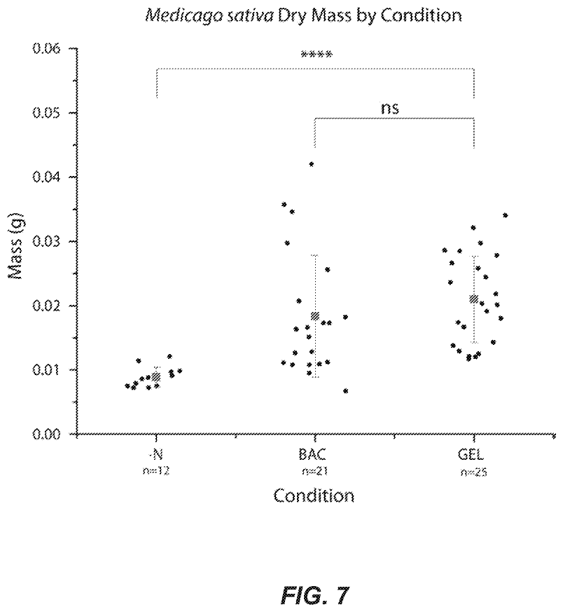

Silica gel carrier effect on alfalfa plant growth. Greenhouse growth experiments demonstrated that the silica gel carrier was able to transport viable bacteria to the alfalfa plants, as shown by confocal imaging of an active S. meliloti Rm1021 nodule present on the inoculated plants (FIG. 5). At the termination of the six-week experiment, the shoot lengths (FIG. 6 & Table 2) of the control plants deprived of nitrogen (—N) averaged 4.6±0.6 cm, the bacterized plants (BAC) averaged 8±2 cm, and the plants with the silica gel carrier (GEL) averaged 8±2 cm. The average shoot length of the plants with the silica gel carrier was 80% longer than the average shoot length of plants deprived of nitrogen sources. Throughout the plant assay, the shoot length of the bacterized plants and the plants inoculated with the silica gel carrier remained similar and never significantly deviated. The average dry mass (FIG. 7) was 9±2 mg for the control plants, 18±9 mg for the bacterized plants, and 21±7 mg for the plants treated with the silica gel carrier. The average dry mass of the plants inoculated with the silica gel carrier was 140% of the average dry mass of the plants deprived of nitrogen sources. Furthermore, no nodules were found on the plants deprived of nitrogen, 12±7 nodules were found on bacterized plants, and 15±8 nodules were found on plants treated with the silica gel carrier. These results indicate that immobilized bacteria were able to be released from the carrier and successfully colonize the root system of the alfalfa plants.

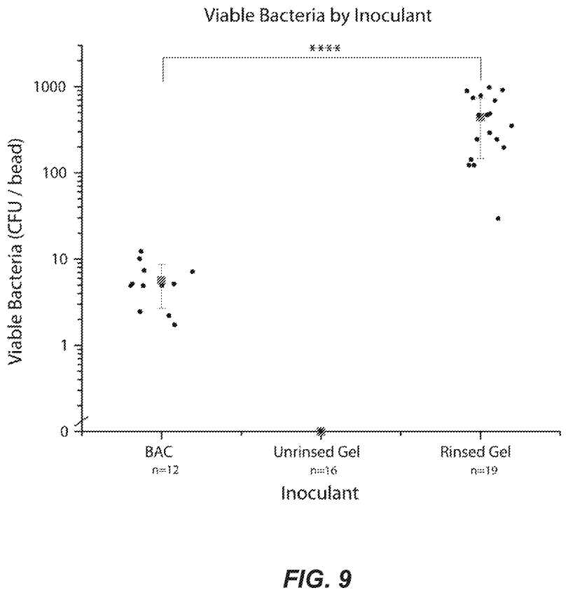

Prior to rinsing the silica gel carrier twice for 20 minutes with PBS of pH 7.0, as described in the Methods section, the viability of unrinsed silica gel was measured. This unrinsed carrier was found to contain 0 CFU/bead (FIG. 9) as a result of the high salt concentration that remained after gelation and subsequent drying. These results illustrated the importance of rinsing the gel after formation to lower salt concentrations in the gel.

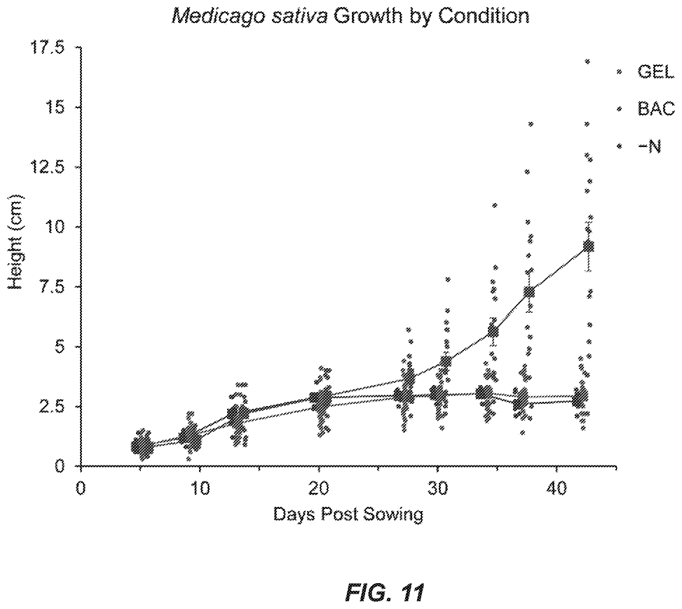

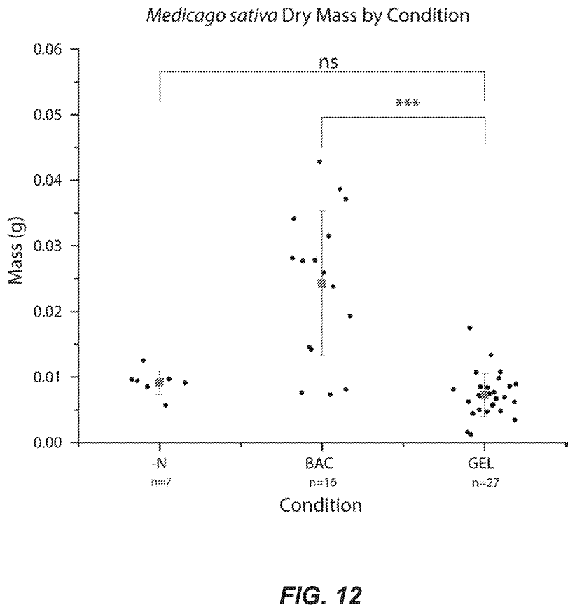

A second plant assay was conducted with the unrinsed silica gel carrier. At the termination of this plant assay, the shoot lengths (FIG. 11) of the control plants deprived of nitrogen (—N) averaged 2.7±0.1 cm, the bacterized plants (BAC) averaged 9±1 cm, and the plants with the silica gel carrier (GEL) averaged 2.9±0.2 cm. The difference in shoot length of the bacterized plants deviated from the other plants by day 27. The average dry mass (FIG. 12) was 9±2 mg for the control plants deprived of nitrogen, 30±20 mg for the bacterized plants, and 7±3 mg for the plants with the silica gel carrier. With regard to both shoot length and dry mass, the silica gel carrier performed similarly to the control group, indicating that the silica gel carrier is biocompatible with the alfalfa plants and not a nutrient source for the alfalfa seeds. This plant assay also demonstrated that maintaining viability of the S. meliloti strain while immobilized was essential to enable the symbiotic relationship with M. sativa.

As shown in Table 1 below, Medicago sativa plants inoculated with the unrinsed silica gel carrier (GEL) exhibited no statistically significant (P=0.47) difference in shoot length from plants deprived of nitrogen sources (—N).

TABLE 1
Medicago sativa plants inoculated with the unrinsed silica
gel carrier (GEL) encapsulating S. meliloti Rm1021

| | Average Height (cm) | | |
|---|---|---|---|
| Days Post Sowing | —N | BAC | GEL |
| 5 | 0.8 ± 0.1 | 0.8 ± 0.1 | 0.8 ± 0.1 |
| 9 | 1.2 ± 0.1 | 1.1 ± 0.1 | 1.3 ± 0.1 |
| 13 | 2.2 ± 0.2 | 2.2 ± 0.2 | 1.8 ± 0.2 |
| 20 | 2.9 ± 0.1 | 2.9 ± 0.2 | 2.5 ± 0.2 |
| 27 | 3.0 ± 0.1 | 3.7 ± 0.2 | 2.9 ± 0.2 |
| 30 | 3.0 ± 0.1 | 4.4 ± 0.4 | 3.0 ± 0.2 |
| 34 | 3.0 ± 0.1 | 5.6 ± 0.6 | 3.1 ± 0.2 |

As shown in Table 2 below, Medicago sativa plants inoculated with the rinsed silica gel carrier exhibited a statistically significant (P=3.2×10−6) difference in shoot length from plants deprived of nitrogen sources after six weeks.

TABLE 2
Medicago sativa plants inoculated with the unrinsed silica
gel carrier (GEL) encapsulating S. meliloti Rm1021

| | Average Height (cm) | | |
|---|---|---|---|
| Days Post Sowing | —N | BAC | GEL |
| 5 | 0.8 ± 0.1 | 0.8 ± 0.1 | 0.8 ± 0.1 |
| 9 | 1.2 ± 0.1 | 1.1 ± 0.1 | 1.3 ± 0.1 |
| 13 | 2.2 ± 0.2 | 2.2 ± 0.2 | 1.8 ± 0.2 |
| 20 | 2.9 ± 0.1 | 2.9 ± 0.2 | 2.5 ± 0.2 |
| 27 | 3.0 ± 0.1 | 3.7 ± 0.2 | 2.9 ± 0.2 |
| 30 | 3.0 ± 0.1 | 4.4 ± 0.4 | 3.0 ± 0.2 |
| 34 | 3.0 ± 0.1 | 5.6 ± 0.6 | 3.1 ± 0.2 |

PGPB have the potential to increase crop yields in an environmentally sustainable and affordable manner. A novel delivery method of PGPB was developed using the sol-gel process to produce silica gel microbeads that encapsulate PGPB and can be applied directly to crop seeds. Through conducting in vitro experiments with alfalfa seeds, it was determined that seeds treated with gel-encapsulated bacteria displayed an order of magnitude increase in bacteria holding capacity relative to bacterized seeds. Furthermore, greenhouse plant assays revealed that the silica gel carrier was able to deliver viable bacteria to the alfalfa plant and increase plant growth by 140% relative to plants deprived of nitrogen.

Utilizing rinsed silica gel carriers, a plant assay provided insight into the effectiveness of the carrier in delivering PGPB. The plants with the silica gel carrier greatly outperformed plants that were deprived of nitrogen; their shoot lengths were on average 80% longer and the average dry mass was 140% greater. The experiment demonstrated the capability of the S. meliloti Rm1021 strain to fix nitrogen, form symbiotic relations with the alfalfa root system, and increase plant growth (both shoot length and dry mass).

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
(1) Bashan, Y.; de-Bashan, L. E.; Prabhu, S. R. Superior Polymeric Formulations and Emerging Innovative Products of Bacterial Inoculants for Sustainable Agriculture and the Environment. In Agriculturally Important Microorganisms: Commercialization and Regulatory Requirements in Asia; Singh, H. B., Sarma, B. K., Keswani, C., Eds.; Springer Singapore: Singapore, 2016; pp 15-46.
(2) Viets, F. G.; Lunin, J. The Environmental Impact of Fertilizers. C R C Critical Reviews in Environmental Control 1975, 5 (4), 423-453.
(3) Tilman, D.; Balzer, C.; Hill, J.; Befort, B. L. Global Food Demand and the Sustainable Intensification of Agriculture. PNAS 2011, 108 (50), 20260-20264.
(4) Bashan, Y.; Hernandez, J.-P.; Leyva, L. A.; Bacilio, M. Alginate Microbeads as Inoculant Carriers for Plant Growth-Promoting Bacteria. Biol Fertil Soils 2002, 35 (5), 359-368.
(5) Nowak, J. Benefits of in Vitro “Biotization” of Plant Tissue Cultures with Microbial Inoculants. In Vitro Cell Dev Biol Plant 1998, 34 (2), 122-130.
(6) Bashan, Y.; de-Bashan, L. E.; Prabhu, S. R.; Hernandez, J.-P. Advances in Plant Growth-Promoting Bacterial Inoculant Technology: Formulations and Practical Perspectives (1998-2013). Plant Soil 2014, 378 (1), 1-33.
(7) Stephens, J. H. G.; Rask, H. M. Inoculant Production and Formulation. Field Crops Research 2000, 65 (2), 249-258.
(8) Bashan, Y. Alginate Beads as Synthetic Inoculant Carriers for Slow Release of Bacteria That Affect Plant Growth. Appl Environ Microbiol 1986, 51 (5), 1089-1098.
(9) Alvarez, G.; Pieckenstain, F.; Desimone, M.; Estrella, M.; Ruiz, O.; Diaz, L. Evaluation of Sol-Gel Silica Matrices as Inoculant Carriers for Mesorhizobium Spp. Cells. In Current Research, Technology and Education Topics in Applied Microbiology and Microbial Biotechnology; Méndez-Vilas, A., Ed.; Formatex, 2010; pp 160-167.
(10) Coiffier, A.; Coradin, T.; Roux, C.; Bouvet, O. M. M.; Livage, J. Sol-Gel Encapsulation of Bacteria: A Comparison between Alkoxide and Aqueous Routes. J. Mater. Chem. 2001, 11 (8), 2039-2044.
(11) Finnie, K. S.; Waller, D. J.; Perret, F. L.; Krause-Heuer, A. M.; Lin, H. Q.; Hanna, J. V.; Barbé, C. J. Biodegradability of Sol-Gel Silica Microparticles for Drug Delivery. J Sol-Gel Sci Technol 2009, 49 (1), 12-18.
(12) Gonçalves, M. C. Sol-Gel Silica Nanoparticles in Medicine: A Natural Choice. Design, Synthesis and Products. Molecules 2018, 23 (8).
(13) John, R. P.; Tyagi, R. D.; Brar, S. K.; Surampalli, R. Y.; Prévost, D. Bio-Encapsulation of Microbial Cells for Targeted Agricultural Delivery. Critical Reviews in Biotechnology 2011, 31 (3), 211-226.
(14) Cheng, H. P.; Walker, G. C. Succinoglycan Is Required for Initiation and Elongation of Infection Threads during Nodulation of Alfalfa by Rhizobium Meliloti. J. Bacteriol. 1998, 180 (19), 5183-5191.
(15) Amaya-Gómez, C. V.; Hirsch, A. M.; Soto, M. J. Biofilm Formation Assessment in Sinorhizobium Meliloti Reveals Interlinked Control with Surface Motility. BMC Microbiology 2015, 15 (1), 58.
(16) Hoagland, D. R.; Arnon, D. I. The Water-Culture Method for Growing Plants without Soil; Berkeley, Calif.: College of Agriculture, University of California, 1950.
(17) Blondeau, M.; Coradin, T. Living Materials from Sol-Gel Chemistry: Current Challenges and Perspectives. J. Mater. Chem. 2012, 22 (42), 22335-22343.
(18) Ferrer, M. L.; Garcia-Carvajal, Z. Y.; Yuste, L.; Rojo, F.; del Monte, F. Bacteria Viability in Sol-Gel Materials Revisited: Cryo-SEM as a Suitable Tool To Study the Structural Integrity of Encapsulated Bacteria. Chem. Mater. 2006, 18 (6), 1458-1463.
(19) Glick, B. R. Plant Growth-Promoting Bacteria: Mechanisms and Applications. Scientifica 2012, 2012.
(20) Lugtenberg, B.; Kamilova, F. Plant-Growth-Promoting Rhizobacteria. Annu. Rev. Microbiol. 2009, 63 (1), 541-556.

What is claimed is:

1. A method of increasing one or more plant growth characteristics in a plant, the method comprising:
(a) providing to a plant seed a solution comprising sodium silicate, citric acid, colloidal silica, and one or more plant growth-promoting bacteria; and
(b) rinsing the plant seed.

2. The method of claim 1, wherein (b) comprises rinsing the plant seed with water.

3. The method of claim 1, wherein (b) comprises rinsing the plant seed with phosphate buffered saline.

4. The method of any of claims 1-3, wherein the solution comprises between 1.0 and 2.0 mol/L sodium silicate.

5. The method of claim 4, wherein the solution comprises about 1.5 mol/L sodium silicate.

6. The method of any of claims 1-5, wherein the solution comprises between 0.5 and 1.5 mol/L citric acid.

7. The method of claim 6, wherein the solution comprises about 1.0 mol/L citric acid.

8. The method of any of claims 1-7, wherein the solution comprises a ratio of the colloidal silica to the sodium silicate of between 2.5:4 and 3.5:4.

9. The method of claim 8, wherein the solution comprises a ratio of the colloidal silica to the sodium silicate of 3:4.

10. The method of any of claims 1-9, wherein the solution is at a pH of between 6.5 and 7.5.

11. The method of claim 10, wherein the solution is at a pH of about 7.0.

12. The method of any of claims 1-11, wherein the solution comprises at least about 10⁷ CFU/mL of the plant growth-promoting bacteria.

13. The method of any of claims 1-12, wherein the solution does not comprise glycerol.

14. The method of any of claims 1-13, wherein (a) comprises aerosolizing the solution to form droplets.

15. The method of claim 14, wherein the droplets are less than 400 μm in diameter.

16. The method of claim 15, wherein the droplets are between about 50 μm and about 200 μm in diameter.

17. The method of any of claims 14-16, wherein the droplets comprise between 10% and 20% silica by weight.

18. The method of claim 17, wherein the droplets comprise about 13% silica by weight.

19. The method of any of claims 14-18, further comprising, prior to (b), allowing the droplets to gel to form silica microbeads.

20. The method of any of claims 1-19, wherein (b) is performed for at least 15 minutes.

21. The method of any of claims 1-20, wherein (b) is performed at least 5 minutes after (a).

22. The method of any of claims 1-21, further comprising rinsing the plant seed a second time.

23. The method of any of claims 1-22, further comprising, prior to (a), generating the solution by mixing (i) a silica solution comprising the sodium silicate, citric acid, and colloidal silica; and (ii) a bacteria solution comprising the plant growth-promoting bacteria.

24. The method of claim 23, wherein the solution does not gel less than 2 minutes following mixing the silica solution and the bacteria solution.

25. The method of claim 24, wherein the solution does not gel less than 3 minutes following mixing the silica solution and the bacteria solution.

26. The method of claim 25, wherein the solution does not gel less than 4 minutes following mixing the silica solution and the bacteria solution.

27. The method of any of claims 1-26, wherein the one or more plant growth-promoting bacteria comprise one or more plant-growth promoting bacteria of Table 1 or Table 2.

28. The method of any of claims 1-26, wherein the one or more plant growth-promoting bacteria comprise Paenibacillus pabuli 151 (NRRL Accession No. B-67417), Dietzia cinnamea 55 (NRRL Accession No. B-67422), Lysinobacillus sphaericus 47 (NRRL Accession No. B-67423), Paenibacillus MBEV37 B17 (Accession No. B-67419), Exiguobacterium alkaliphilum 20 (NRRL Accession No. B-67425), Bacillus safensis 34 (NRRL Accession No. B-67620), Methylobacterium dankookense Bots301 (NRRL Accession No. B-67981), Methylobacterium radiotolerans Bots303 (NRRL Accession No. B-67982), Fictibacillus phosphorivorans Bots309 (NRRL Accession No. B-67983), Bradyrhizobium japonicum USDA 110, Sinorhizobium meliloti Rm1021, or Micromonospora sp. UTRUM1 (NRRL Accession No. B-67418).

29. The method of claim 28, wherein the one or more plant growth-promoting bacteria comprise Sinorhizobium meliloti Rm1021.

30. The method of claim 29, wherein the one or more plant growth-promoting bacteria comprise Bradyrhizobium japonicum USDA 110.

31. The method of any of claims 1-30, wherein the plant seed is a dicotyledon plant seed, a crop plant seed, or a legume plant seed.

32. The method of claim 31, wherein the plant seed is an alfalfa plant seed.

33. The method of any of claims 1-32, further comprising planting the plant seed.

34. A method of making a population of plant-growth promoting silica microbeads, the method comprising:
(a) aerosolizing a solution comprising sodium silicate, citric acid, colloidal silica, and one or more plant-growth promoting bacteria to generate droplets;
(b) allowing the droplets to gel to form silica microbeads; and
(c) rinsing the silica microbeads.

35. The method of claim 34, wherein (c) comprises rinsing the silica microbeads with water.

36. The method of claim 34, wherein (c) comprises rinsing the silica microbeads with phosphate buffer saline.

37. The method of any of claims 34-36, wherein the solution comprises between 1.0 and 2.0 mol/L sodium silicate.

38. The method of claim 37, wherein the solution comprises about 1.5 mol/L sodium silicate.

39. The method of any of claims 34-38, wherein the solution comprises between 0.5 and 1.5 mol/L citric acid.

40. The method of claim 39, wherein the solution comprises about 1.0 mol/L citric acid.

41. The method of any of claims 34-40, wherein the solution comprises a ratio of the colloidal silica to the sodium silicate of between 2.5:4 and 3.5:4.

42. The method of claim 41, wherein the solution comprises a ratio of the colloidal silica to the sodium silicate of 3:4.

43. The method of any of claims 34-42, wherein the solution is at a pH of between 6.5 and 7.5.

44. The method of claim 43, wherein the solution is at a pH of about 7.0.

45. The method of any of claims 34-44, wherein the solution comprises at least about 107 CFU/mL of the plant growth-promoting bacteria.

46. The method of any of claims 34-45, wherein the solution does not comprise glycerol.

47. The method of any of claims 34-46, wherein the droplets are less than 400 μm in diameter.

48. The method of claim 47, wherein the droplets are between about 50 μm and about 200 μm in diameter.

49. The method of any of claims 34-48, wherein the droplets comprise between 10% and 20% silica by weight.

50. The method of claim 49, wherein the droplets comprise about 13% silica by weight.

51. The method of any of claims 34-50, wherein (c) is performed for at least 15 minutes.

52. The method of any of claims 34-51, wherein (c) is performed at least 5 minutes after (b).

53. The method of any of claims 34-52, further comprising rinsing the silica microbeads a second time.

54. The method of any of claims 34-53, further comprising, prior to (a), generating the solution by mixing (i) a silica solution comprising the sodium silicate, citric acid, and colloidal silica; and (ii) a bacteria solution comprising the plant growth-promoting bacteria.

55. The method of claim 54, wherein the droplets do not gel less than 2 minutes following mixing the silica solution and the bacteria solution.

56. The method of claim 55, wherein the droplets do not gel less than 3 minutes following mixing the silica solution and the bacteria solution.

57. The method of claim 56, wherein the droplets do not gel less than 4 minutes following mixing the silica solution and the bacteria solution.

58. The method of any of claims 34-57, wherein the one or more plant growth-promoting bacteria comprise one or more plant-growth promoting bacteria of Table 1 or Table 2.

59. The method of any of claims 34-57, wherein the one or more plant growth-promoting bacteria comprise Paenibacillus pabuli 151 (NRRL Accession No. B-67417), Dietzia cinnamea 55 (NRRL Accession No. B-67422), Lysinobacillus sphaericus 47 (NRRL Accession No. B-67423), Paenibacillus MBEV37 B17 (Accession No. B-67419), Exiguobacterium alkaliphilum 20 (NRRL Accession No. B-67425), Bacillus safensis 34 (NRRL Accession No. B-67620), Bradyrhizobium japonicum USDA 110, Sinorhizobium meliloti Rm1021, or Micromonospora sp. UTRUM1 (NRRL Accession No. B-67418).

60. A plant seed coated with a population of silica microbeads comprising at least 1000 colony forming units (CFU) of plant-growth promoting bacteria.

61. The plant seed of claim 60, wherein the plant seed comprises at least 3000 CFU of the plant-growth promoting bacteria.

62. The plant seed of claim 61, wherein the plant seed comprises at least 5000 CFU of the plant-growth promoting bacteria.

63. The plant seed of claim 62, wherein the plant seed comprises at least 10000 CFU of the plant-growth promoting bacteria.

64. The plant seed of claim 63, wherein the plant seed comprises at least 15000 CFU of the plant-growth promoting bacteria.

65. The plant seed of any of claims 60-64, wherein the one or more plant growth-promoting bacteria comprise one or more plant-growth promoting bacteria of Table 1 or Table 2.

66. The plant seed of any of claims 60-64, wherein the plant growth-promoting bacteria is Paenibacillus pabuli 151 (NRRL Accession No. B-67417), Dietzia cinnamea 55 (NRRL Accession No. B-67422), Lysinobacillus sphaericus 47 (NRRL Accession No. B-67423), Paenibacillus MBEV37 B17 (Accession No. B-67419), Exiguobacterium alkaliphilum 20 (NRRL Accession No. B-67425), Bacillus safensis 34 (NRRL Accession No. B-67620), Bradyrhizobium japonicum USDA 110, Sinorhizobium meliloti Rm1021, or Micromonospora sp. UTRUM1 (NRRL Accession No. B-67418).

67. The plant seed of claim 66, wherein the plant growth-promoting bacteria is Bradyrhizobium japonicum USDA 110.

68. The plant seed of claim 66, wherein the plant growth-promoting bacteria is Sinorhizobium meliloti Rm1021.

69. The plant seed of any of claims 60-68, wherein the population of silica microbeads comprises at least 50 silica microbeads.

70. The plant seed of claim 69, wherein the population of silica microbeads comprises at least 100 silica microbeads.

71. The plant seed of claim 70, wherein the population of silica microbeads comprises at least 500 silica microbeads.

72. The plant seed of claim 71 wherein the population of silica microbeads comprises at least 1000 silica microbeads.

73. The plant seed of any of claims 60-72, wherein the plant seed is a dicotyledon plant seed, a crop plant seed, or a legume plant seed.

74. The plant seed of any of claims 60-72, wherein the plant seed is an alfalfa plant seed.

75. A population of plant seeds comprising the plant seed of any of claims 60-74.

76. A silica microbead encapsulating one or more plant growth-promoting bacteria.

77. The silica microbead of claim 76, wherein the microbead has a diameter of less than 400 μm.

78. The silica microbead of claim 77, wherein the microbead has a diameter of less than 200 μm.

79. The silica microbead of claim 78, wherein the microbead has a diameter of less than 100 μm.

80. The silica microbead of claim 79, wherein the microbead has a diameter of about 50 μm.

81. The silica microbead of any of claims 76-80, wherein the microbead is between 10% and 20% silica by weight.

82. The silica microbead of claim 81, wherein the microbead is about 13% silica by weight.

83. The silica microbead of any of claims 76-82, wherein the one or more plant growth-promoting bacteria comprise Paenibacillus pabuli 151 (NRRL Accession No. B-67417), Dietzia cinnamea 55 (NRRL Accession No. B-67422), Lysinobacillus sphaericus 47 (NRRL Accession No. B-67423), Paenibacillus MBEV37 B17 (Accession No. B-67419), Exiguobacterium alkaliphilum 20 (NRRL Accession No. B-67425), or Bacillus safensis 34 (NRRL Accession No. B-67620), Bradyrhizobium japonicum USDA 110, Sinorhizobium meliloti Rm1021, or Micromonospora sp. UTRUM1 (NRRL Accession No. B-67418).

84. The silica microbead of claim 83, wherein the one or more plant growth-promoting bacteria comprise Sinorhizobium meliloti Rm1021.

85. The silica microbead of claim 83, wherein the one or more plant growth-promoting bacteria comprise Bradyrhizobium japonicum USDA 110.

86. A population of microbeads comprising the silica microbead of any of claims 76-85.

87. The population of microbeads of claim 86, wherein the population of microbeads comprises at least about 10⁷ CFU of the plant growth promoting bacteria.

88. The population of microbeads of claim 86 or 87, wherein the population of microbeads comprises an additional silica microbead encapsulating an additional plant growth-promoting bacteria.

89. A plant seed coated with the population of microbeads of any of claims 86-88.

90. The plant seed of claim 89, wherein the plant seed is a dicotyledon plant seed, a crop plant seed, or a legume plant seed.

91. The plant seed of claim 90, wherein the plant seed is an alfalfa plant seed.

92. A method of increasing one or more plant growth characteristics in a plant, the method comprising providing to the plant an effective amount of (i) the silica microbead of any of claims 76-85 or (ii) the population of microbeads of any of claims 86-86.
